(19) United States
(12) Patent Application Publication
(10) Pub. No.: US 2021/0345962 A1
(43) Pub. Date: Nov. 11, 2021

(54) REMOTE REHABILITATION SYSTEM

(71) Applicant: City of Hope, Duarte, CA (US)

(72) Inventors: Yuman Fong, Duarte, CA (US); Lily L. Lai, Duarte, CA (US); Jennifer Hayter, Duarte, CA (US); Sherry Hite, Duarte, CA (US); Jamie Rand, Duarte, CA (US)

(21) Appl. No.: 17/313,899

(22) Filed: May 6, 2021

Related U.S. Application Data
(60) Provisional application No. 63/021,305, filed on May 7, 2020.

(51) Int. Cl. A61B 5/00; A61B 5/11

(52) U.S. Cl. CPC A61B 5/6804; A61B 5/1121; A61B 5/0004; A61B 2505/09; A61B 5/6825; A61B 2562/0219; A61B 5/6823

(57) ABSTRACT

Methods and systems are described for a remote rehabilitation system. The system includes at least one garment configured to be worn by a user, a plurality of sensors coupled to the at least one garment, and the plurality of sensors arranged to measure a range of motion of the user, and a controller communicatively coupled to the plurality of sensors and configured to receive data from the plurality of sensors. The controller is configured to measure the range of motion of the user wearing the at least one garment based on the data received from the plurality of sensors. Additionally, the controller is configured to determine whether the range of motion satisfies a threshold.

### TECHNICAL FIELD

The present disclosure relates generally to occupational therapy, and more particularly, to a remote rehabilitation system.

### BACKGROUND

Rehabilitation includes exercises to improve mobility and strength. Rehabilitation accelerates surgery recovery and the return to normal activities. Occupational therapy and rehabilitation are needed for recovering cancer survivors. For example, breast cancer survivors face difficulties maintaining strength and health due to chemotherapy, radiation, and surgery. Rehabilitation reverses the debilitating effects of chemotherapy, radiation, and surgery in recovering breast cancer survivors.

Additionally, rehabilitation prevents complications and chronic illness for cancer survivors. For example, breast cancer survivors face long-term complications from breast surgery include lymphedema, neurologic pain, and axillary web syndrome. Chronic illness results in limitations in range of motion, diminished strength, and activity restrictions. This ultimately translates to reduced quality of life for cancer survivors. The medical community has emphasized the importance of exercise that monitors or increases range of motion, strength, and bodily activity to prevent these chronic illnesses. For example, the National Lymphedema Network recommends providing exercises that track baseline physical abilities to identify lymphedema at the earliest possible stage for recovering breast cancer patients. Monitoring physical health and rehabilitation participation reduces the incidents of health impairments, improves health outcomes, and reduces health care costs.

Currently, occupational and physical therapy and rehabilitation services may not be accessible due to high medical costs, shortage of occupational and physical therapists, and geographic constraints. Moreover, health care personnel may be unable to closely supervise treatment, potentially allowing a patient to perform below their physical abilities. More worrisome, health care personnel may be unable to identify a chronic illness at the earliest possible stage. As such, these care and supervision limitations lead to chronic illness and reduced quality of life.

### SUMMARY

The present disclosure provides methods, systems, articles of manufacture, including computer program products, for a remote rehabilitation system.

In one aspect, there is provided a system including at least one garment configured to be worn by a user. The system includes a plurality of sensors coupled to the at least one garment, the plurality of sensors arranged to measure a range of motion of the user. The system includes a controller communicatively coupled to the plurality of sensors and is configured to receive data from the plurality of sensors. The controller is configured to measure the range of motion of the user wearing the at least one garment based on the data received from the plurality of sensors. The controller is also configured to determine, in response to measuring the range of motion of the user wearing the at least one garment, whether the range of motion satisfies a threshold.

In some variations, measuring the range of motion of the user wearing the at least one garment includes measuring an angle of a user limb to determine a minimum angle and a maximum angle. The controller is further configured to determine whether the user is performing the exercise along a predetermined trajectory based on the data received from the plurality of sensors. In some variations, the controller is further configured to determine a type of exercise being performed by the user based on the data received from the plurality of sensors. The controller is further configured to determine a number of repetitions performed by the user associated with the exercise based on the data received from the plurality of sensors. The range of motion is associated with an exercise performed by the user. Additionally, the number of repetitions performed by the user is determined by maximum angles detected using a peak detection function.

In some variations, the plurality of sensors are further configured to monitor an upright orientation of the user. In some variations, the plurality of sensors includes a back sensor configured to be positioned near a back of the user. The controller is further configured to determine a back angle based on the data received from the back sensor. The controller is further configured to determine whether the back angle satisfies a back angle threshold, the back angle threshold indicative of the user slouching while performing an exercise.

Additionally, the plurality of sensors includes a hand sensor configured to be positioned at or near a dorsum of a hand of the user. The controller is configured to determine hand orientation of the user based on data received from the dorsum of the hand sensor, the hand orientation indicative of whether the user is performing an exercise as prescribed. In some variations, the controller is further configured to present a notification via a user interface based on the range of motion satisfying the threshold, and wherein the range of motion is indicative of a level of effort by the user.

In some variations, the plurality of sensors includes a first sensor configured to be a reference sensor, and wherein the plurality of sensors includes at least one of an accelerometer, a gyroscope, and an Inertial Measurement Unit (IMU). In some variations, a wireless communication interface coupled to the at least one garment, the wireless communication interface communicatively coupled to the plurality of sensors and configured to transmit the data received from the plurality of sensors.

In another aspect, there is provided a device including at least one garment configured to be worn by a user. The device includes a plurality of sensors coupled to the at least one garment, the plurality of sensors arranged to measure a range of motion of the user. The device includes a wireless communication interface coupled to the at least one garment, the wireless communication interface communicatively coupled to the plurality of sensors and configured to transmit data received from the plurality of sensors. In some variations, the plurality of sensors includes a first sensor configured to be a reference sensor, and wherein the plurality of sensors includes at least one of an accelerometer, a gyroscope, and an Inertial Measurement Unit (IMU).

Implementations of the current subject matter may include methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which may include a non-transitory computer-readable or machine-readable storage medium, may include, encode, store, or the like one or more programs that cause one or more processors to perform one or more of the operations described herein. Computer-implemented methods consistent with one or more implementations of the current subject matter may be implemented by one or more data processors residing in a single computing system or multiple computing systems.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes, it should be readily understood that such features are not intended to be limiting. The claims that follow this disclosure are intended to define the scope of the protected subject matter.

### BRIEF DESCRIPTION OF THE DRAWINGS

The embodiments herein may be better understood by referring to the following description in conjunction with the accompanying drawings in which like reference numerals indicate identically or functionally similar elements, of which:

FIG. 1 depicts an example of a garment including a plurality of sensors for a remote rehabilitation system;

FIG. 2 depicts an example of a block diagram for a remote rehabilitation system;

FIG. 3A depicts an example of a sensor for a remote rehabilitation system;

FIG. 3B depicts an example of a plurality of sensors for a remote rehabilitation system;

FIG. 4 depicts an example of a secondary microcontroller with a communication interface for a remote rehabilitation system;

FIG. 5A depicts an example of a remote rehabilitation system measuring the range of motion of a user;

FIG. 5B depicts an example of a graph illustrating the range of motion of the user measured by the remote rehabilitation system;

FIG. 6 depicts an example of a remote rehabilitation system measuring the number of repetitions performed by the user associated with an exercise;

FIG. 7A depicts an example of a back sensor measuring the range of motion of a user in an upright position;

FIG. 7B depicts an example of a back sensor measuring the range of motion of a user in a slouched position;

FIG. 7C depicts an example of a graph illustrating the back angle of the user measured by the back sensor;

FIG. 8 depicts an example of a chart illustrating the hand orientation of the user measured by the remote rehabilitation system;

FIG. 9 depicts an example of a user interface for the remote rehabilitation system;

FIG. 10A depicts an example of a user interface of the remote rehabilitation system for displaying progress;

FIG. 10B depicts another example of a user interface of the remote rehabilitation system for displaying progress;

FIG. 10C depicts an example of a user interface of the remote rehabilitation system for determining user health;

FIG. 11 depicts an example of a user interface of the remote rehabilitation system for determining rehabilitation goals; and

FIG. 12 depicts a block diagram illustrating a computing system consistent with implementations of the current subject matter.

### DETAILED DESCRIPTION

The remote rehabilitation system presents a telemedicine solution to breast cancer patients requiring occupational or physical therapy and preventative treatment. The remote rehabilitation system may prevent complications and chronic illnesses. For example, the remote rehabilitation system may prevent the long-term complications of lymphedema, neurologic pain, and axillary web syndrome in breast cancer survivors. The remote rehabilitation system may provide occupational therapy and other rehabilitative therapy services to breast cancer patients who have difficulty accessing medical services due to high costs, a shortage of rehabilitative therapists, or geographic constraints. The remote rehabilitation system may accelerate breast cancer patient recovery, mobility, and activities due to its on-demand availability.

The remote rehabilitation system may monitor and supervise physical exercises at a greater level of attention than medical professionals. For example, the remote rehabilitation system may closely supervise user movements to identify small deviations in performance indicative of a chronic illness at the earliest stages. In contrast, medical professionals may only identify greater deviations in user performance once the chronic illness has reached a later stage. Accordingly, the remote rehabilitation system prevents chronic illness and leads to greater quality of life for breast cancer patients.

According to the present disclosure, the remote rehabilitation system may include a garment, a plurality of sensors coupled to the garment, and a controller coupled to the plurality of sensors. The garments may be configured to be worn by a user. The plurality of sensors may be arranged to measure a range of motion of the user. The controller may be configured to receive data from the plurality of sensors. The controller may also be configured to measure the range of motion of the user wearing the garment based on the data received from the plurality of sensors. The controller may be configured to determine whether the range of motion satisfies a threshold.

The range of motion may be associated with an exercise performed by the user. To measure the range of motion, the controller may measure an angle of a user limb to determine a minimum angle and a maximum angle. The controller may be configured to present a notification via a user interface based on the range of motion satisfying the threshold. The controller may determine whether the user is performing the exercise along a predetermined trajectory based on the data received from the plurality of sensors. The controller may determine a type of exercise being performed by the user based on the data received from the plurality of sensors.

In some embodiments, the plurality of sensors may further be configured to monitor an upright orientation of the user. The plurality of sensors may include a back sensor configured to be positioned near the back of the user. The controller may be configured to determine a back angle based on the data received from the back sensor. The controller may be configured to determine whether the back angle satisfies a back angle threshold where the back angle threshold is indicative of the user slouching while performing an exercise.

In some embodiments, the plurality of sensors may include a hand sensor configured to be positioned near the hand of the user. The controller may be configured to determine hand orientation of the user based on data received from the hand sensor. The hand orientation may be indicative of whether the user is performing an exercise as prescribed. The controller may determine the number of repetitions performed by the user associated with the exercise based on the data received from the plurality of sensors. To determine the number of repetitions, the controller may detect the maximum angles using a peak detection function. The controller may determine the effort generated by the user based on the data received by the plurality of sensors.

A remote rehabilitation system solves technical problems associated with measuring a range of motion in breast cancer patients. Sensors coupled to the garment are configured to measure user range of motion and performance of exercises to a higher degree of accuracy than otherwise achievable by humans or other hardware implementations. For example, imprecise human measurements may increase the risk for a false negative of maintained or improved range of motion, leading to delayed detection of lymphedema in breast cancer survivors. In contrast, the specific arrangement of sensors coupled to the garment in the remote rehabilitation system accurately detects posture and range of motion, eliminating false negatives that stunt breast cancer survivor rehabilitation and early illness detection.

Additionally, the unique arrangement of sensors of the remote rehabilitation system improves on existing hardware implementations. For example, other devices may measure a range of motion based on a sensor or camera detached from the user. Such detached measurements lead to inaccuracies as these devices are limited by their ability to capture only relative measurements and are unable to consistently perceive and accommodate differences in user posture, limb angles, or changes in body size. In contrast, the remote rehabilitation system utilizes a unique arrangement of sensors attached to a garment that are configured to perceive user posture, limb angles, and body positions. Further, the controller, when communicatively coupled to the sensors, is configured to factor in changes to user posture, limb angles, and body positioning to obtain an accurate range of motion measurements. The unique combination of the garment, the plurality of sensors, and controller configurations overcome the failure of older technologies.

The methods, systems, apparatuses, and non-transitory storage mediums described herein operate the remote rehabilitation system to measure a range of motion and to determine whether the range of motion satisfies a threshold. The various exemplary embodiments also disclose a wearable device including a garment, a plurality of sensors coupled to the garment, and a wireless communication interface coupled to the garment.

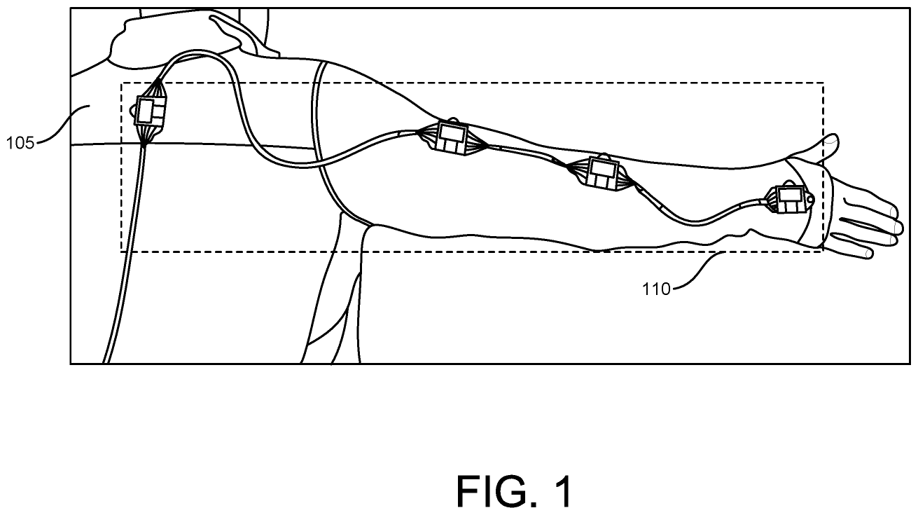

FIG. 1 depicts an example of a garment 105 including a plurality of sensors 110 for a remote rehabilitation system. The garment 105 is configured to be worn by the user. The plurality of sensors 110 may be coupled to the garment 105. For example, the sensors 110 may be sewn into the garment 105 and may be interconnected by wires in the garment 105. Additionally, and/or alternatively, the sensors 110 may be removably coupled to the garment 105. For example, the sensors 110 may be selectively detached from the garment 105 via a fastener, tie, button, zipper, velcro, pocket, and/or the like.

The garment 105 may be a wearable article of clothing. For example, the garment 105 may be a long sleeve jacket configured to cover the user chest, back, and arms. The garment 105 may be pants, an arm sleeve, a hat, a helmet, a chest band, an arm band, a glove, a brace, a dress, a wrap, a sleeveless garment, and/or the like. Additionally, and/or alternatively, the garment 105 may be configured to cover a portion of a limb of the user. The garment 105 may include hard-wired connections sewn into the garment 105 to communicatively couple the plurality of sensors 110. The garment 105 may include wires that are selectively attached to the garment 105.

The garment 105 may include a hole in the sleeve or use other attachments of the garment to position the sensors correctly. Positioning of the sensors accurately may prevent the garment 105 and the sensors 110 from twisting around the user. In at least one embodiment, a jacket may have a hole at the end of each sleeve through which the user thumb is inserted while worn. The hole at the end of the sleeve may prevent the displacement of the sensors 110 along the sleeve. Additionally, the hole at the end of the sleeve may secure the thumb for accurate measurement of thumb orientation.

The garment 105 may be slightly compressive. The garment 105 may be sufficiently tight to ensure the sensors 110 remain in contact with the user. In some implementations, a jacket may compress around the user arm and back to ensure that sensors 110 remain in contact with the user. The garment 105 may be manufactured from materials that enable the user to move freely.

A plurality of sensors 110 may be fastened to the garment 105. The sensors 110 may be arranged to measure the relevant movements of the user. For example, sensors 110 measuring arm movement may be fastened to a long sleeve jacket at the upper arm, the forearm, and the hand. The upper arm, the forearm, and the hand may be strategic locations for the placement of the sensors 110 as they capture relevant movements of the arm. The sensors 110 may be configured to measure a range of motion. In some embodiments, the sensors 110 may measure arm movement with three sensors 110 placed along the arm and one centered at the upper back. The sensors 110 may be configured to capture movement for a particular user exercise. For example, the sensors 110 may be configured to measure front arm raises and side arm raises. The sensors 110 may be configured to simultaneously measure the range of motion of multiple limbs. For example, the sensors 110 may be configured to measure side arm raises for both the right and left arms.

In some implementations, the first sensor may provide a frame of reference for the other sensors. The other sensors may use the first sensor as a point of reference to measure user movement. In some exemplary embodiments, sensors located at an upper arm, the forearm, and the hand may use the first sensor as a point of reference. The first sensor placed at the upper portion of the back may measure patient posture, including front-to-back leaning (slouching) as well as side-to-side leaning. The sensors 110 may be rearranged for different occupational therapy exercises.

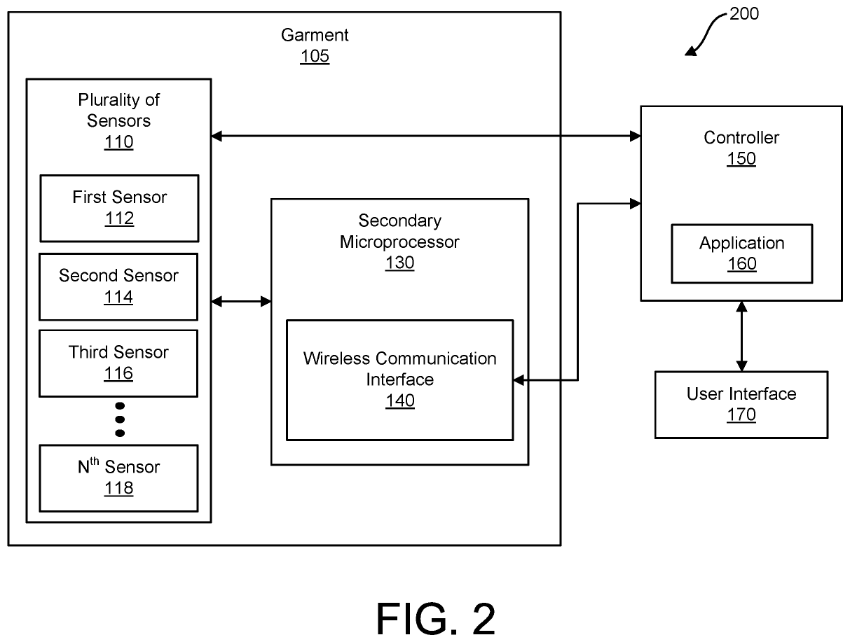

FIG. 2 depicts an example of a block diagram for a remote rehabilitation system. The remote rehabilitation system 200 may include a garment 105, a plurality of sensors 110 coupled to the garment 105, and a controller 150 communicatively coupled to the plurality of sensors 110. The controller 150 may be communicatively coupled to a computing device with a user interface 170. The controller 150 may be operable to run application 160. In some embodiments, the remote rehabilitation system 200 may include a secondary microcontroller 130 and a wireless communication interface 140.

The plurality of sensors 110 may be arranged to collect data for measuring a range of motion of the user. The plurality of sensors 110 may include an inertial measurement unit, an accelerometer, a gyroscope, and a magnetometer. The accelerometer may be configured to measure proper acceleration (including gravity) along the x, y, and z coordinate axes. The gyroscope may be configured to measure angular rotation rates around the x, y, and z coordinate axes. The magnetometer may be configured to measure the magnetic field strength along the x, y, and z coordinate axes. The plurality of sensors 110 may transmit quaternion data from each of the sensors to the controller 150. For example, the sensors 110 may detect movement and transmit the movement as versors for representing spatial orientations and rotations of elements in three-dimensional space.

The plurality of sensors 110 may be communicatively coupled to a secondary microcontroller 130. The secondary microcontroller 130 may perform preliminary data processing, such as filtering quaternion data from the plurality of sensors 110. The secondary microcontroller 130 may be coupled to the garment 105 and be configured to receive data from the plurality of sensors 110. The plurality of sensors 110 may be communicatively coupled to a wireless communication interface 140. The wireless communication interface 140 may be coupled to the garment 105 and be configured to transmit the data received from the plurality of sensors 110. For example, the plurality of sensors 110 may be communicatively coupled a Bluetooth interface coupled to the garment 105 for transmitting data received from the plurality of sensors 110.

The controller 150 may be configured to receive data from the plurality of sensors 110. For example, the controller 150 may be configured to receive the data from the plurality of sensors 110 via the wireless communication interface 140. The controller 150 may be configured to determine the range of motion of the user wearing the garment 105 based on the data received from the plurality of sensors 110. The controller 150 may be configured to determine whether the range of motion satisfies a threshold. The controller 150 may be at a computing device detached from the garment 105, such as a mobile device, a computer, and/or the like. Alternatively, and/or additionally, the controller 150 may be coupled to the garment 105.

The controller 150 may transform the quaternion data from the sensors 110 to body frame data using matrix transformations. The transformed quaternion data may be turned into roll, pitch, and yaw data in reference to the user, the floor, or other points of reference that may include sensors on the garment. Roll, pitch, and yaw data may respectively represent the rotations around the x, y, and z axes. The controller 150 may determine the exercise being performed, how many repetitions are performed, the range of the motion of the patient, and whether the patient is doing each of the exercises along a predetermined trajectory and in a predetermined form based on the roll, pitch, and yaw data.

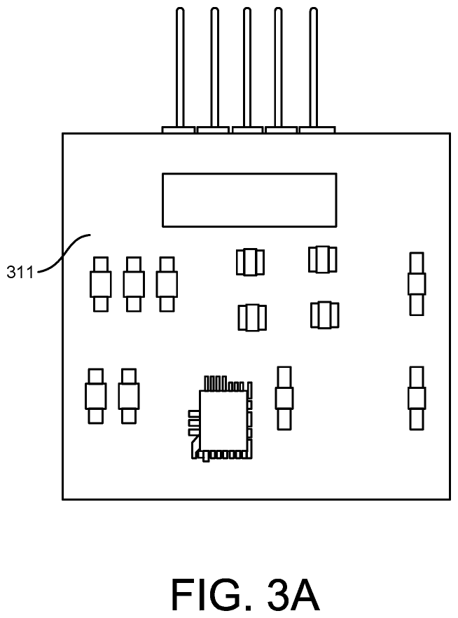

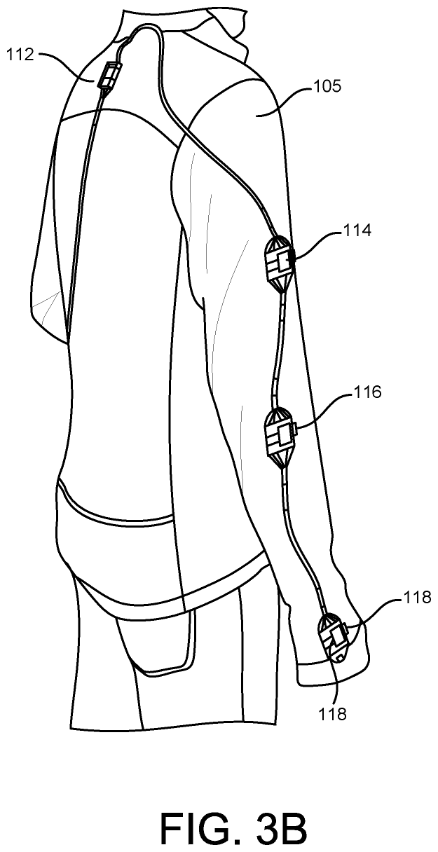

FIG. 3A depicts an example of a sensor for a remote rehabilitation system. The sensor 311 may include an accelerometer, a gyroscope, and a magnetometer. The sensor 311 may report measurements along three perpendicular axes (x, y, and z axes). The accelerometer may be configured to measure proper acceleration (including gravity) along the x, y, and z coordinate axes. The gyroscope may be configured to measure angular rotation rates around the x, y, and z coordinate axes. The magnetometer may be configured to measure the magnetic field strength along the x, y, and z coordinate axes. The sensor 311 may generate quaternion data representing spatial orientation and rotation of the sensor 311 in three-dimensional space. The sensor 311 may include a 3D digital linear acceleration sensor, a 3D digital angular rate sensor, or a 3D digital magnetic sensor. In at least one embodiment, the inertial measurement units may generate versors representing spatial orientations and rotations of elements in three-dimensional space.

FIG. 3B depicts an example of a plurality of sensors for a remote rehabilitation system. The plurality of sensors 110 may include an inertial measurement unit, an accelerometer, a gyroscope, and a magnetometer. The plurality of sensors 110 may be coupled to the garment 105. For example, the sensors 110 may be sewn into the garment 105 and may be interconnected by wires in the garment 105. Additionally, and/or alternatively, the sensors 110 may be removably coupled to the garment 105. For example, the sensors 110 may be selectively detached from the garment 105 via a fastener, tie, button, zipper, velcro, pocket, and/or the like. The sensors 110 may be communicatively coupled via an Inter-Integrated Circuit interface or an SPI interface. The sensors 110 may be communicatively coupled via a wireless interface.

The sensors 110 may be arranged to measure the relevant movements of the user. For example, sensors 110 may be arranged on a long sleeve jacket at the upper arm (e.g., second sensor 114), the forearm (e.g., second sensor 116), and the hand (e.g., N^{th} sensor 118). The upper arm, the forearm, and the hand may be strategic locations for the placement of the sensors as they capture relevant movements of the arm. The sensors 110 may be configured to measure range of motion. In some embodiments, the sensors 110 may measure arm range of motion with three sensors placed along an arm and one centered at the upper back. The sensors 110 may be configured to capture movement for a particular user exercise. For example, the sensors 110 may be configured to measure front arm raises and side arm raises. The sensors 110 may be configured to simultaneously measure the range of motion of multiple limbs. For example, the sensors 110 may be configured to measure side arm raises for both the right and left arms.

In some implementations, the first sensor 112 may provide a frame of reference for the other sensors. The other sensors may use the first sensor as a point of reference to measure user movement. In some exemplary embodiments, sensors located at the upper arm (e.g., second sensor 114), the forearm (e.g., second sensor 116), and the hand (e.g., N^{th} sensor 118) may use the first sensor 112 as a point of reference.

A first sensor 112 may be placed on the upper back portion of the garment 105. The first sensor 112 may measure patient posture, including front-to-back leaning (slouching) as well as side-to-side leaning. For example, the first sensor 112 may measure the angle of the first sensor at the upper back relative to the floor. The other sensors may be placed along the length of a user arm to measure the movement of each arm segment. In some exemplary embodiments, the other sensors may be located proximate to the upper arm (e.g., second sensor 114), the forearm (e.g., second sensor 116), and the hand (e.g., N^{th} sensor 118) for measuring the movement of each arm segment. The sensors 110 may be rearranged for different occupational therapy exercises.

In some implementations, sensors 110 may be fastened to pants at the thigh, the calf, and the foot for measuring leg movements. The thigh, the calf, and the foot may be strategic locations for the placement of the sensors 110 as they capture relevant movements of the leg. The sensors 110 may be configured to measure a leg range of motion. In some exemplary arrangements, the sensors 110 may measure leg movement with three sensors placed along the leg and one centered at the lower back. The sensors 110 may be configured to capture movement for a particular user exercise. For example, the sensors 110 may be configured to measure front leg extensions and side leg extensions. The sensors 110 may be configured to simultaneously measure the range of motion of multiple legs. For example, the sensors 110 may be configured to measure side leg extensions for both the left and right legs.

In some implementations, a first sensor 112 may be placed on the lower back portion of the garment 105. The first sensor 112 may provide a frame of reference for the other sensors. The first sensor 112 may measure patient posture, including front-to-back leaning (slouching) as well as side-to-side leaning. For example, the first sensor 112 may measure the angle of the first sensor 112 at the lower back relative to the floor. The other sensors may be placed along the length of a user leg to measure the movement of each leg segment. For example, the other sensors may be located proximate to the thigh (e.g., second sensor 114), the calf (e.g., third sensor), and the foot (e.g., N^{th} sensor).

In some embodiments, the sensors 110 may be arranged such that relevant movements of the user hand and palm can be measured by the sensors 110. For example, sensors 110 may be fastened to a glove at the thumb, index finger, middle finger, ring finger, pinky finger, dorsum, and palm. The thumb, index finger, middle finger, ring finger, pinky finger, dorsum, and palm may be strategic locations for the placement of the sensors 110 as they capture relevant movements of the hand. The sensors 110 may be configured to measure range of motion. In some embodiments, the sensors 110 may measure hand movement with sensors placed at the fingers, the wrist, dorsum, and the palm. The sensors 110 may be configured to capture movement for a particular user exercise. For example, the sensors 110 may be configured to measure front arm raises and side arm raises. In another example, the sensors 110 at the hand may be configured to determine whether the palm orientation is correct for a particular exercise. The hand orientation may be indicative of whether the user is performing an exercise as prescribed. The sensors 110 may be configured to simultaneously measure the range of motion of multiple hands. For example, the sensors 110 may be configured to measure side arm raises for both the right and left hands. In some embodiments, a hand sensor may be placed at or near a dorsum of a hand of the user. The controller may be configured to determine hand orientation of the user based on data received from the hand sensor. The hand orientation may be indicative of whether the user is performing an exercise as prescribed.

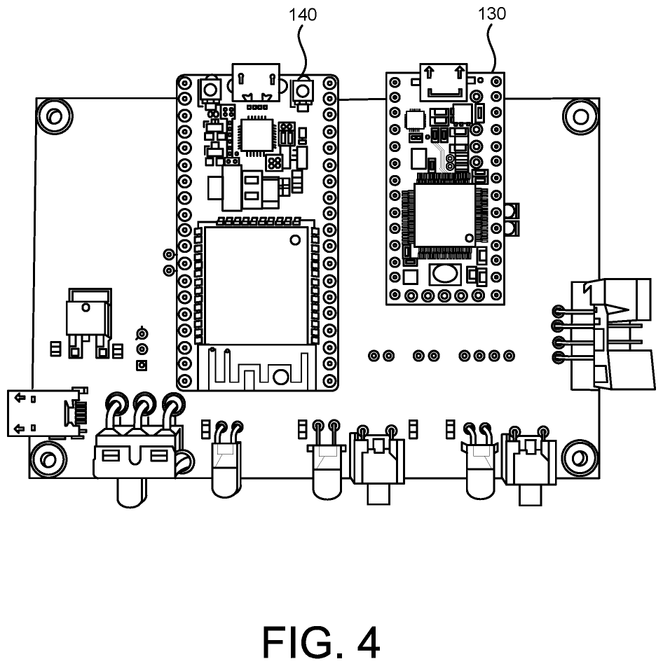

FIG. 4 depicts an example of a secondary microcontroller with a communication interface for a remote rehabilitation system. The secondary microcontroller 130 may be communicatively coupled to the plurality of sensors 110. The secondary microcontroller 130 may perform preliminary data processing, such as filtering quaternion data from the plurality of sensors 110. The secondary microcontroller 130 may be coupled to the garment 105 and be configured to receive data from the plurality of sensors 110. The secondary microcontroller 130 may be communicatively coupled to a wireless communication interface 140. The plurality of sensors 110 may be communicatively coupled to a wireless communication interface 140. The wireless communication interface 140 may be coupled to the garment 105 and be configured to transmit the data received from the plurality of sensors 110. For example, the plurality of sensors 110 may include a Bluetooth interface coupled to the garment 105 for transmitting data received from the plurality of sensors 110.

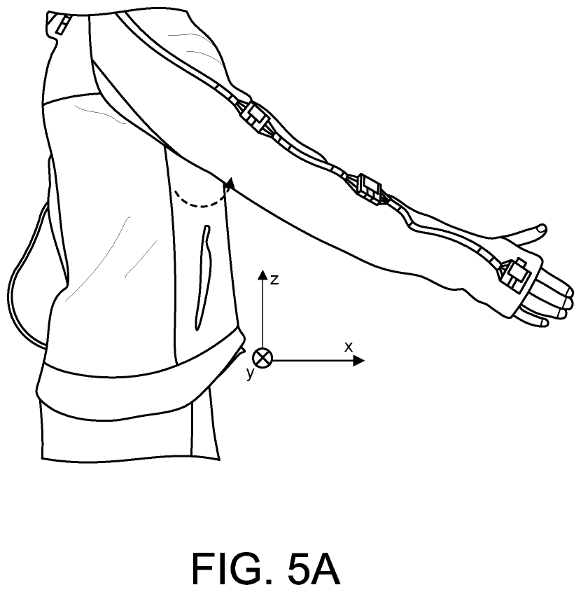

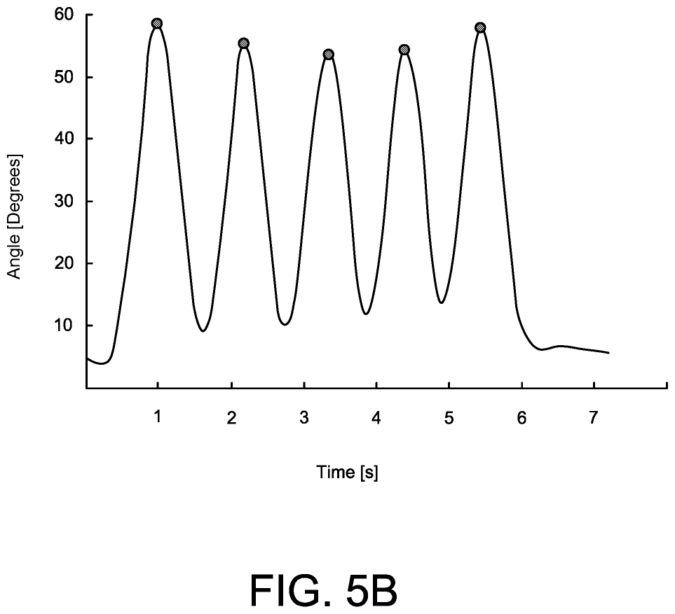

FIG. 5A depicts an example of a remote rehabilitation system measuring the range of motion of a user. A controller 150 may be communicatively coupled to the wireless communication interface 140. The controller 150 may be communicatively coupled to the plurality of sensors 110. The controller 150 may be communicatively coupled to a computing device, such as a mobile device, with a user interface 170 and operable to run application 160. The controller 150 may be at a computing device detached from the garment 105, such as a mobile device, a computer, and/or the like. Alternatively, and/or additionally, the controller 150 may be coupled to the garment 105.

The controller 150 may be configured to determine the range of motion of the user wearing the garment 105 based on the data received from the plurality of sensors 110. The controller 150 may determine the range of motion by measuring an angle of a user limb to determine a minimum angle and a maximum angle. For example, the controller 150 may determine the user has an arm range of motion by determining a lower limit and an upper limit of the range of motion. The lower limit of the arm range of motion may be measured as an arm resting position down by the user's side. The upper limit of the arm range of motion may be measured as the arms extended above the user shoulders. In another example, the lower limit of the arm range of motion may be measured as the arms extended 20 degrees behind the user back. The upper limit of the arm range of motion may be measured as the arms extended 15 degrees in front of the user head.

The controller 150 may be configured to determine whether the range of motion satisfies a threshold. The threshold may be satisfied with respect to the floor, the user, or a sensor. The range of motion threshold may be satisfied by the user reaching an upper limit. For example, the user may satisfy an upper limit of arm range of motion by extending their arm 30 degrees with respect to the floor. In another example, the user may satisfy an upper limit of arm range of motion by extending their arm 25 degrees behind the back sensor. The range of motion threshold may be satisfied by the user reaching a lower limit. For example, the user may satisfy a lower limit of their arm range of motion by extending their arm 10 degrees behind the user back. In another example, the user may satisfy a lower limit of their arm range of motion by extending their arm 5 degrees behind the sensor at the user upper arm. The range of motion threshold may be calculated by subtracting the lower limit from the upper limit. For example, the upper limit of 30 degrees with respect to the floor and a lower limit of 10 degrees behind the user back satisfies a range of motion threshold of 130 degrees. In another example, the upper limit of 40 degrees with respect to the first sensor and a lower limit of 25 degrees behind the user back satisfies a range of motion threshold of 155 degrees.

In some embodiments, the controller 150 may receive a signal or parameter indicating what arrangement of sensors 110 is the lower limit. For example, the controller 150 may receive a signal generated by user input indicating that −95 degrees with respect to the x-axis is the lower limit. In another example, the controller 150 may receive a parameter indicating −80 degrees with respect to the x-axis is the lower limit. In some embodiments, the controller 150 may receive a signal or parameter indicating what arrangement of sensors 110 is the upper limit. For example, the controller 150 may receive a signal generated by user input indicating that 5 degrees with respect to the x-axis is the upper limit. In another example, the controller 150 may receive a parameter indicating that 45 degrees with respect to the x-axis is the upper limit.

The controller 150 may determine whether the user is performing the exercise along a predetermined trajectory based on the data received from the plurality of sensors 110. For example, the controller 150 may monitor the x, y, and z axis movements to determine the user is extending arms to the side according to predetermined trajectories for side arm raises. The controller 150 may generate a warning in response to detecting the user does not extend arms directly to the side. In another example, the controller 150 may monitor the x, y, and z axis movements to determine that the user maintains an upright posture as the user lifts a weight to their chest. The controller 150 may generate a warning in response to determining that the back angle satisfies a predetermined threshold. In another example, the controller 150 may monitor the x, y, and z axis movements to determine the knee does not bend while performing a calf stretch. The controller 150 may generate a warning that the knee is bent is in response to a sensor detecting that the knee angle satisfies a threshold. The controller 150 may continue to generate the warning until the knee angle does not satisfy the threshold.

The controller 150 may determine a type of exercise being performed by the user based on the data received from the plurality of sensors 110. For example, the controller 150 may determine that side arm raises are performed based on the movement of the arm sensors in the y-axis. In another example, the controller 150 may determine that the user is stretching their legs based on the leg sensors being in a horizontal configuration along the x-axis. In response to detecting the type of exercise being performed by the user, the controller 150 may display instructions and provide feedback regarding the correct performance of the exercise.

The controller 150 may transform the quaternion data from the sensors to body frame data using matrix transformations. The transformed quaternion data may be turned into roll, pitch, and yaw data with reference to the user. Roll, pitch, and yaw data may respectively represent the rotations around the x, y, and z axes. Roll, pitch, and yaw data may determine the exercise being performed, how many repetitions are performed, the range of the motion of the patient, and whether the patient is doing each of the exercises along a predetermined trajectory and in a predetermined form.

Application 160 may include instructions to display program modes and user sessions related to tracking range of motion for various users. The application 160 may include instructions to configure the presentation of notifications, counters, tutorials, goals, user health, progress, and selectable options at the user interface 170. The application 160 may include instructions to configure the organization of notifications, counters, tutorials, goals, user health, progress, and selectable options at the user interface 170. In some embodiments, the application 160 may include machine learning or artificial intelligence to monitor the progress of the user. The artificial intelligence may monitor the effort of the user based on past performance, the range of motion measured by the sensors, and the status of the user rehabilitation.

FIG. 5B depicts an example of a graph illustrating the range of motion of the user measured by the remote rehabilitation system. The graph may depict the user range of motion over several repetitions. For example, the graph may display the arm angle over time using data collected from the hand sensor during the front arm raises exercise. The peaks may correspond to the upper limit of the user movement. The troughs may correspond to the lower limit of the user movement. The controller 150 may determine the threshold was satisfied by the peaks satisfying a predetermined angle or the troughs satisfying a predetermined angle. The controller 150 may determine the threshold was satisfied by subtracting the troughs from the peaks. Additionally, and/or alternatively, the controller 150 may determine the range of motion by using a peak detection function.

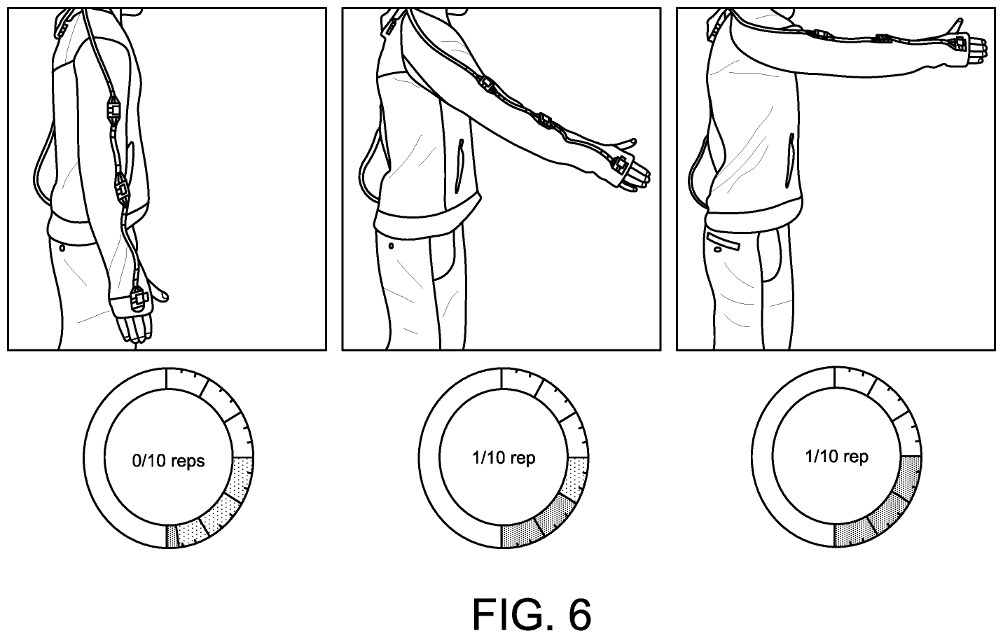

FIG. 6 depicts an example of a remote rehabilitation system measuring the number of repetitions performed by the user associated with an exercise. The controller 150 may determine the number of repetitions performed by the user associated with the exercise based on the data received from the plurality of sensors 110. The controller 150 may determine the number of repetitions performed by the user based on the number of maximum angles detected using a peak detection function. Additionally, and/or alternatively, the controller 150 may determine the number of repetitions completed based on pitch data from the sensors 110.

The controller 150 may determine to track the number of repetitions for the user via the user interface 170. The controller 150 may present the number of repetitions completed by the user through the user interface 170 and the goal number of repetitions to be completed by the user. The controller 150 may be configured to provide real-time feedback to the user. For example, the controller 150 may provide a graph depicting the position of the user limb (e.g., arm) with respect to the user body. Once the position of the user limb satisfies a threshold, the controller 150 may update the number of repetitions completed by the user.

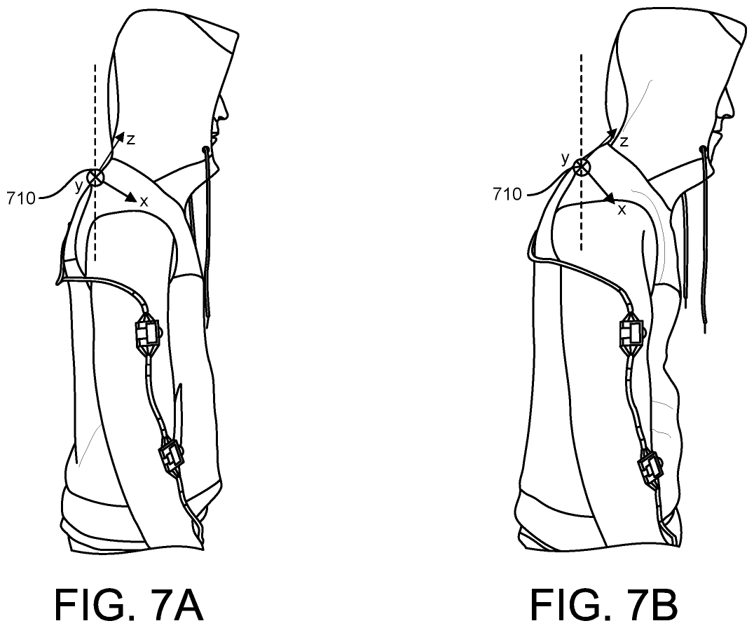

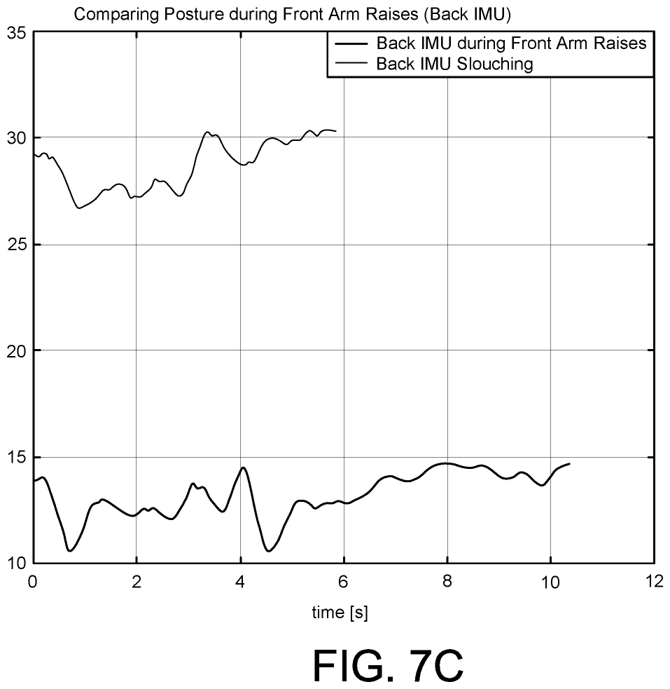

FIG. 7A depicts an example of a back sensor measuring the range of motion of a user in an upright position. The plurality of sensors 110 may include a back sensor 710. The back sensor 710 may be configured to monitor an upright orientation of the user. The back sensor 710 may be placed on the upper back portion or a lower back portion of the garment 105.

The controller 150 may be configured to determine a back angle based on the data received from the back sensor 710. The back sensor 710 may provide a frame of reference for the other sensors. The back sensor 710 may measure patient posture, including front-to-back leaning (slouching) as well as side-to-side leaning (slouching sideways). For example, the back sensor 710 may measure the angle of the back sensor 710 as 70 degrees relative to the floor in the x-direction, which is indicative that the user is slouching. In another example, the back sensor 710 may measure that the angle of the back sensor 710 as 75 degrees relative to the floor in the y-direction. This is indicative that the user is tilted to the side while performing the exercise. Additionally, and or alternatively, the back sensor 710 may determine the back angle relative to the floor, the other sensors, or the user. The user may also be able to set an upright angle. The controller 150 may use the upright angle set by the user to determine that the user has not maintained an upright position.

The controller 150 may be configured to determine whether the back angle satisfies a back angle threshold. The back angle threshold may be indicative of the user slouching while performing an exercise. Satisfying the back angle threshold may determine the user is slouching forward or slouching sideways. The back angle threshold may be satisfied with respect to the floor, the user, or a sensor. The back angle threshold may be determined while the user is standing up straight. The back angle threshold may be determined by measuring the rotation around the y axis while the user is standing up straight. The controller 150 may determine the threshold is satisfied by measuring the rotation around the y axis. The back angle threshold may be satisfied by the user satisfying a lower limit. For example, the user may satisfy the threshold in response to the back sensor 710 measuring an angle of 70 degrees relative to the floor in the x-direction. In another example, the user may satisfy the threshold in response to the back sensor measuring an angle of 75 degrees relative to the floor in the y-direction. The controller 150 may generate a warning when the back angle threshold is satisfied

In some embodiments, the controller 150 may receive a signal or parameter indicating what back angle is the lower limit. For example, the controller 150 may receive a signal generated by user input indicating that 70 degrees relative to a thigh sensor in the x-direction is the lower limit. In another example, the controller 150 may receive a parameter indicating 80 degrees relative to the floor in the y-direction is the lower limit.

FIG. 7B depicts an example of a back sensor measuring the range of motion of a user in a slouched position.

FIG. 7C depicts an example of a graph illustrating the back angle of the user measured by the remote rehabilitation system. The graph may depict the back angle over time. The graph may display the back angle over time using data collected from the back sensor 710 during an exercise. The lower curve in the figure may display the back angle of a person doing the exercise with an upright posture as detected by the back sensor 710. The lower curve data may show the patient baseline back angle is around 10 degrees. The upper curve may display the back angle of a person doing the exercise in a slouched position. In the upper curve, the average back angle is around 25 degrees, which is indicative that the user is in a slouched position similar to the user depicted in FIG. 7B.

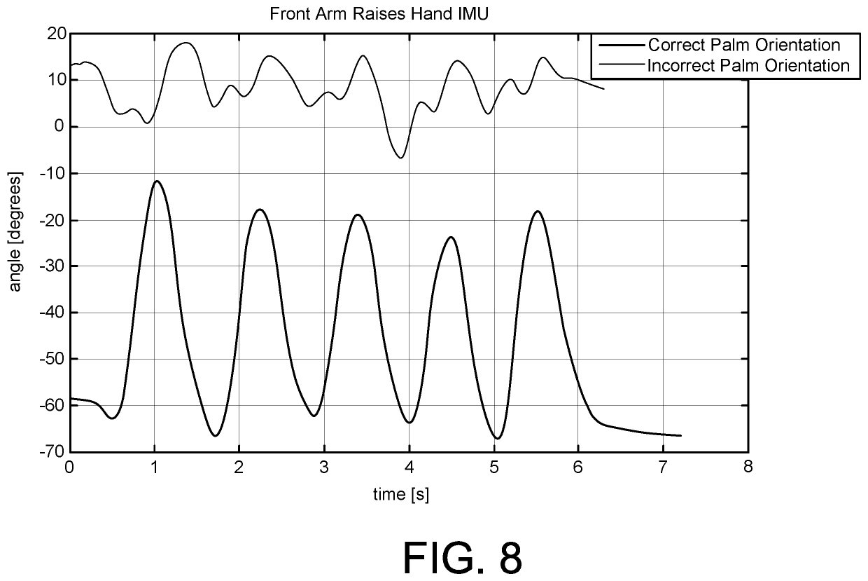

FIG. 8 depicts an example of a chart illustrating the hand orientation of the user measured by the remote rehabilitation system. Hand orientation may be incorrect during rehabilitation exercises. For example, the palm pointed to the floor instead of pointed to the side may be a mistake during a front arm raise.

The controller 150 may determine hand orientation based on data received from a hand sensor. The hand orientation may be indicative of whether the user is performing an exercise as prescribed. For example, the lower curve on the graph may be representative of hand sensor rotation around the y-axis oscillating with an amplitude of around 50 degrees, which may be indicative of the correct hand orientation. In contrast, the upper curve may be representative of an inconsistent hand sensor rotation around the y-axis oscillating with an amplitude of around 10 degrees, which is indicative of an incorrect palm orientation.

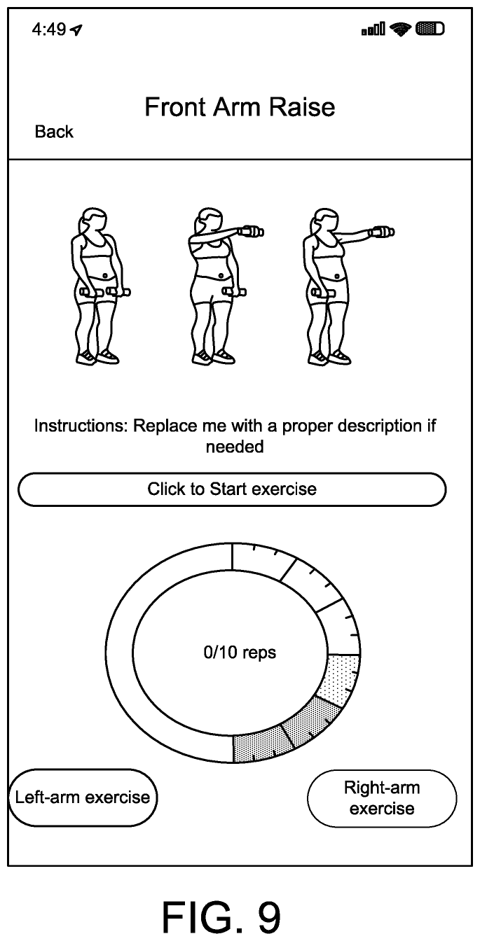

FIG. 9 depicts an example of a user interface for the remote rehabilitation system. The user interface 170 may be at a computer, a mobile device, and/or the like. The user interface 170 may include a touchscreen. The controller 150 may be communicatively coupled to the user interface 170. The user interface 170 may display program modes and user sessions of the application 160.

The controller 150 may be configured to present a notification via a user interface 170 based on the range of motion satisfying a threshold. The controller 150 may be configured to present a notification via a user interface 170 based on the back angle satisfying a back angle threshold. The controller 150 may present an exercise tutorial for the user to perform the exercise via the user interface 170. The controller 150 may present a counter to track the number of repetitions performed by the user via the user interface 170. The controller 150 may present a real-time dial to display the angle of arm movement via the user interface 170. The controller 150 may present a selectable option to determine the type of exercise to be performed. The controller 150 may present how an exercise is performed. The controller 150 may present a prompt to correct the user in response to detecting the sensors 110 do not follow a predetermined trajectory. The application 160 may include instructions to configure the presentation of notifications, counters, tutorials, and selectable options at the user interface 170.

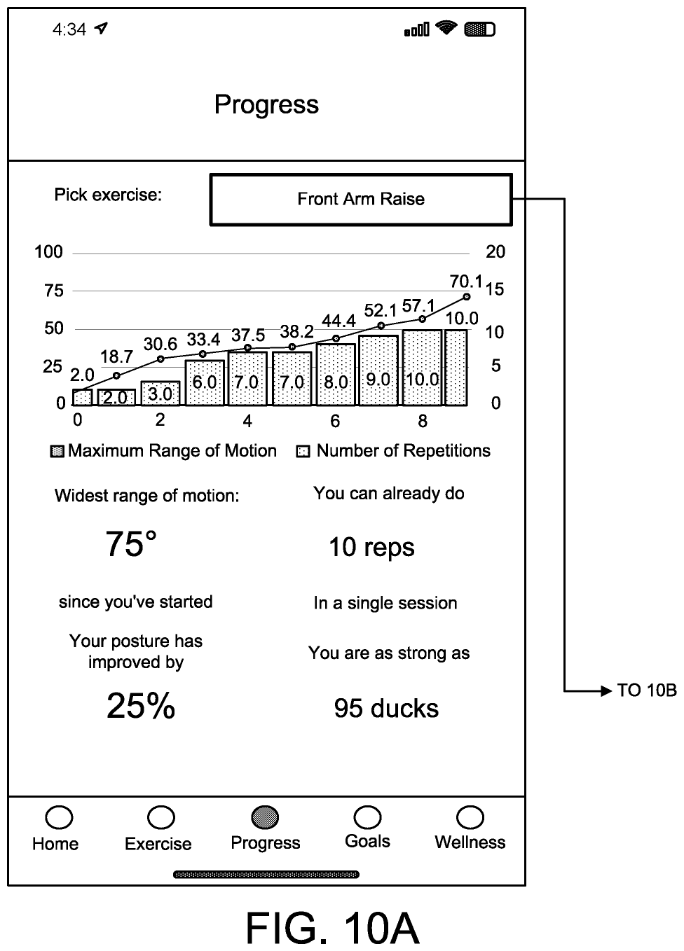

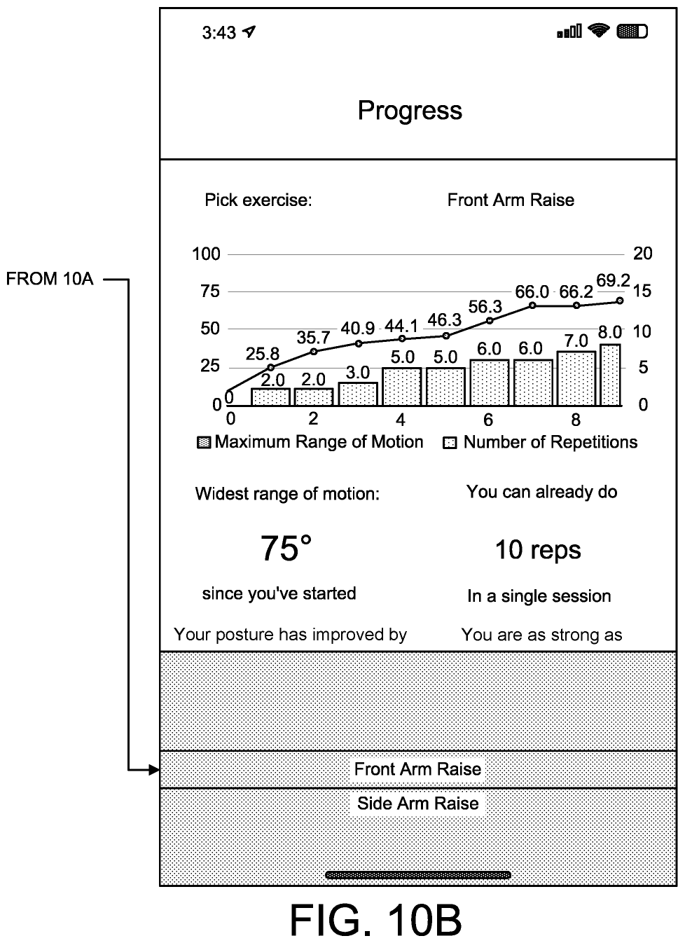

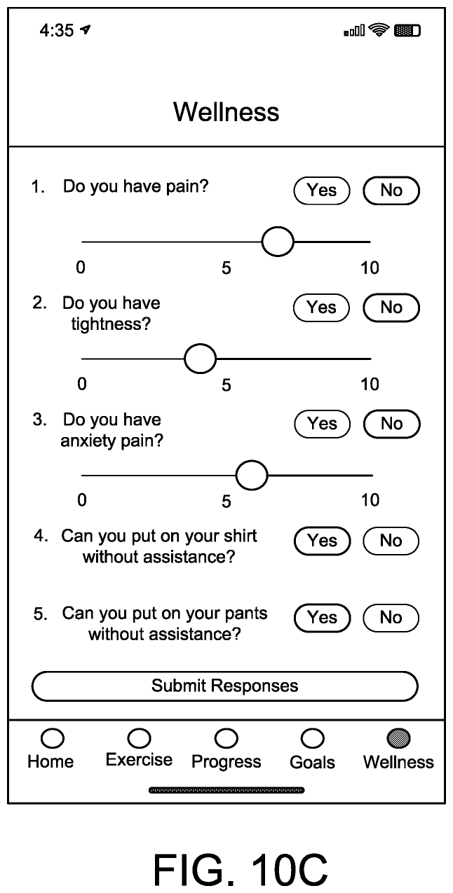

FIG. 10A depicts an example of another user interface of the remote rehabilitation system for displaying progress. The controller 150 may be configured to present progress with respect to the user range of motion via the user interface 170. For example, the controller 150 may present the widest range of motion measured by the plurality of sensors 110. In another example, the controller 150 may present a graph of the measured ranges of motion over time. The controller 150 may be configured to present progress for various exercises. For example, the controller 150 may present a selectable option to enable the user to view progress for front arm raises. Additionally, the controller 150 may generate an exercise program based on the user progress. For example, the controller 150 may generate a more strenuous program for users who have shown progress over the past 10 days. The controller 150 may be configured to present details of user performance from previous user sessions, including when the number of errors that the user made while performing the exercise. The application 160 may include instructions to configure the presentation of progress at the user interface 170. FIG. 10B depicts another example of a user interface of the remote rehabilitation system for displaying progress.

FIG. 10C depicts an example of a user interface of the remote rehabilitation system for determining user health. The controller 150 may be configured to gather data related to progress via a user interface 170. The controller 150 may be configured to present questions and receive user responses related to user pain, user tightness, and user activities to measure progress related to the user range of motion. Additionally, the controller 150 may generate an exercise program based on the user responses. For example, the controller 150 may generate a less strenuous program for users who are unable to dress themselves. The application 160 may include instructions to configure the presentation of user health at the user interface 170.

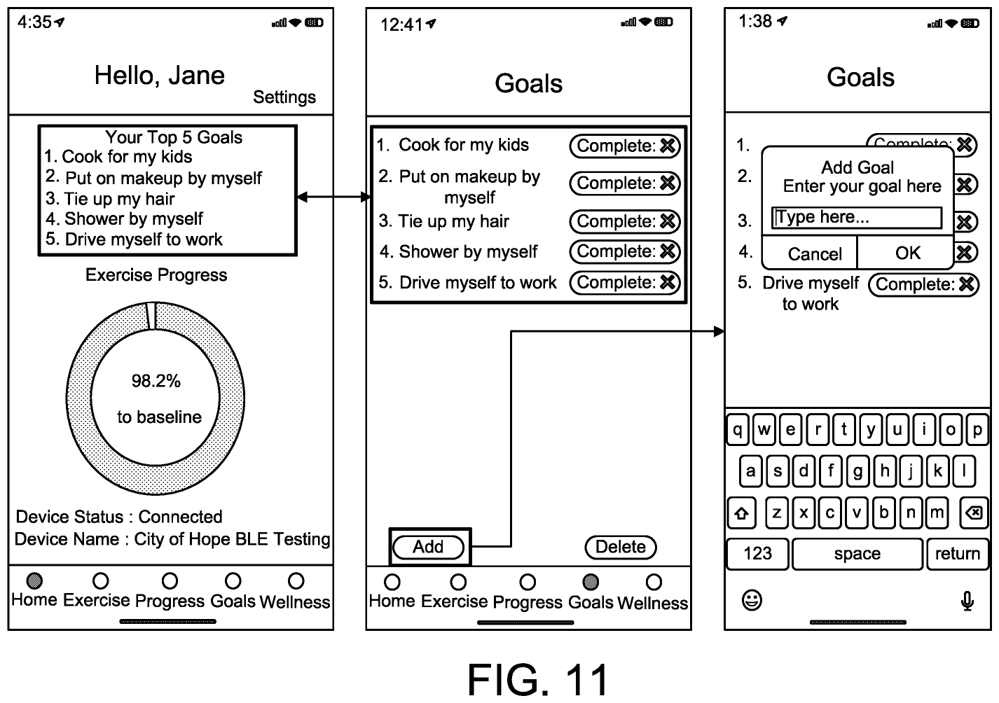

FIG. 11 depicts an example of a user interface of the remote rehabilitation system for determining rehabilitation goals. The controller 150 may be configured to track activities related to progress via a user interface 170. The controller 150 may determine the types of activities necessary for the user to arrive at a baseline of physical health. For example, the controller 150 may determine that the user is 80% to a baseline of physical health by being able to perform 8 out of 10 activities for the past 10 days. The controller 150 may be configured to store goals related the user rehabilitation and update the user baseline of health based on the stored goals. For example, the controller 150 may receive a new goal of being able to drive a car from the user. The controller 150 may update the baseline of the user ability to perform this goal. The application 160 may include instructions to configure the presentation of goals at the user interface 170.

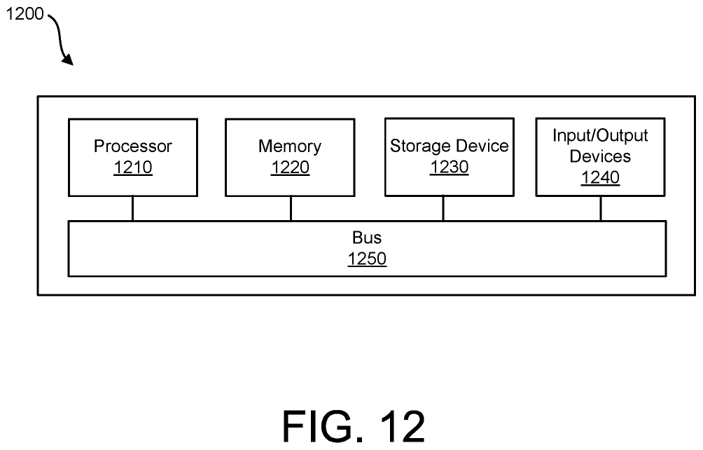

FIG. 12 depicts a block diagram illustrating a computing system 1200 consistent with implementations of the current subject matter. Referring to FIGS. 1-12, the computing system 1200 may enable the remote rehabilitation system. For example, the computing system 1200 may implement user equipment, a personal computer, or a mobile device.

As shown in FIG. 12, the computing system 1200 may include a processor 1210, a memory 1220, a storage device 1230, and an input/output device 1240. The processor 1210, the memory 1220, the storage device 1230, and the input/output device 1240 may be interconnected via a system bus 1250. The processor 1210 is capable of processing instructions for execution within the computing system 1200. Such executed instructions may implement one or more components of, for example, a remote rehabilitation system 200. In some example embodiments, the processor 1210 may be a single-threaded processor. Alternately, the processor 1210 may be a multi-threaded processor. The processor 1210 is capable of processing instructions stored in the memory 1220 and/or on the storage device 1230 to display graphical information for a user interface provided via the input/output device 1240.

The memory 1220 is a non-transitory computer-readable medium that stores information within the computing system 1200. The memory 1220 may store data structures representing configuration object databases, for example. The storage device 1230 is capable of providing persistent storage for the computing system 1200. The storage device 1230 may be a floppy disk device, a hard disk device, an optical disk device, or a tape device, or other suitable persistent storage means. The input/output device 1240 provides input/output operations for the computing system 1200. In some example embodiments, the input/output device 1240 includes a keyboard and/or pointing device. In various implementations, the input/output device 1240 includes a display unit for displaying graphical user interfaces.

According to some example embodiments, the input/output device 1240 may provide input/output operations for a network device. For example, the input/output device 1240 may include Ethernet ports or other networking ports to communicate with one or more wired and/or wireless networks (e.g., a local area network (LAN), a wide area network (WAN), the Internet, a public land mobile network (PLMN), and/or the like).

In some example embodiments, the computing system 1200 may be used to execute various interactive computer software applications that may be used for organization, analysis and/or storage of data in various formats. Alternatively, the computing system 1200 may be used to execute any type of software applications. These applications may be used to perform various functionalities, e.g., planning functionalities (e.g., generating, managing, editing of spreadsheet documents, word processing documents, and/or any other objects, etc.), computing functionalities, communications functionalities, etc. The applications may include various add-in functionalities or may be standalone computing items and/or functionalities. Upon activation within the applications, the functionalities may be used to generate the user interface provided via the input/output device 1240. The user interface may be generated and presented to a user by the computing system 1200 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program item, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) or organic light emitting diode (OLED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

In the following description, for purposes of explanation, numerous specific details are set forth in order to provide a thorough understanding of the various embodiments. It should be understood that other embodiments may be utilized, and structural changes may be made without departing from the scope of the disclosed subject matter. Any combination of the following features and elements is contemplated to implement and practice the disclosure.

In the description, common or similar features may be designated by common reference numbers. As used herein, “exemplary” may indicate an example, an implementation, or an aspect, and should not be construed as limiting or as indicating a preference or a preferred implementation

The many features and advantages of the disclosure are apparent from the detailed specification, and thus, it is intended by the appended claims to cover all such features and advantages of the disclosure which fall within the true spirit and scope of the disclosure. Further, since numerous modifications and variations will readily occur to those skilled in the art, it is not desired to limit the disclosure to the exact construction and operation illustrated and described, and accordingly, all suitable modifications and equivalents may be resorted to, falling within the scope of the disclosure.

What is claimed is:

1. A system comprising:
at least one garment configured to be worn by a user;
a plurality of sensors coupled to the at least one garment, the plurality of sensors arranged to measure a range of motion of the user;
a controller communicatively coupled to the plurality of sensors and configured to receive data from the plurality of sensors, the controller configured to:
measure the range of motion of the user wearing the at least one garment based on the data received from the plurality of sensors; and
determine, in response to measuring the range of motion of the user wearing the at least one garment, whether the range of motion satisfies a threshold.

2. The system of claim 1, wherein measuring the range of motion of the user wearing the at least one garment includes measuring an angle of a user limb to determine a minimum angle and a maximum angle.

3. The system of claim 1, wherein the range of motion is associated with an exercise performed by the user and wherein the controller is further configured to:
determine whether the user is performing the exercise along a predetermined trajectory based on the data received from the plurality of sensors.

4. The system of claim 1, wherein the range of motion is associated with an exercise performed by the user and wherein the controller is further configured to:
determine a type of exercise being performed by the user based on the data received from the plurality of sensors.

5. The system of claim 1, wherein the range of motion is associated with an exercise performed by the user and wherein the controller is further configured to:
determine a number of repetitions performed by the user associated with the exercise based on the data received from the plurality of sensors.

6. The system of claim 5, wherein the number of repetitions performed by the user is determined by maximum angles detected using a peak detection function.

7. The system of claim 1, wherein the plurality of sensors are further configured to monitor an upright orientation of the user.

8. The system of claim 1, wherein the plurality of sensors includes a back sensor configured to be positioned near a back of the user, and wherein the controller is further configured to:
determine a back angle based on the data received from the back sensor; and
determine whether the back angle satisfies a back angle threshold, the back angle threshold indicative of the user slouching while performing an exercise.

9. The system of claim 1, wherein the plurality of sensors includes a hand sensor configured to be positioned at or near a dorsum of a hand of the user, wherein the controller is further configured to:
determine a hand orientation of the user based on data received from the hand sensor, the hand orientation indicative of whether the user is performing an exercise as prescribed.

10. The system of claim 1, wherein the controller is further configured to present a notification via a user interface based on the range of motion satisfying the threshold, and wherein the range of motion is indicative of a level of effort by the user.

11. The system of claim 1, wherein the plurality of sensors includes a first sensor configured to be a reference sensor, and wherein the plurality of sensors includes at least one of an accelerometer, a gyroscope, and an Inertial Measurement Unit (IMU).

12. The system of claim 1, further comprising:
a wireless communication interface coupled to the at least one garment, the wireless communication interface communicatively coupled to the plurality of sensors and configured to transmit the data received from the plurality of sensors,
wherein the controller is communicatively coupled to the wireless communication interface.

13. A non-transitory computer-readable storage medium storing instructions that, when executed by a processor, cause the processor to perform operations comprising:
measuring a range of motion of a user wearing at least one garment based on data received from a plurality of sensors; and
determining, in response to measuring the range of motion of the user wearing the at least one garment, whether the range of motion satisfies a threshold.

14. The non-transitory computer-readable storage medium of claim 13, wherein measuring the range of motion of the user wearing the at least one garment includes measuring an angle of a user limb to determine a minimum angle and a maximum angle.

15. The non-transitory computer-readable storage medium of claim 13, wherein the range of motion is associated with an exercise performed by the user and wherein the operations further comprise:
determining whether the user is performing the exercise along a predetermined trajectory based on the data received from the plurality of sensors.

16. The non-transitory computer-readable storage medium of claim 13, wherein the range of motion is associated with an exercise performed by the user and wherein the operations further comprise:
determining a type of exercise being performed by the user based on the data received from the plurality of sensors.

17. The non-transitory computer-readable storage medium of claim 13, wherein the range of motion is associated with an exercise performed by the user and wherein the operations further comprise:
determining a number of repetitions performed by the user associated with the exercise based on the data received from the plurality of sensors.

18. The non-transitory computer-readable storage medium of claim 17, wherein the number of repetitions performed by the user are determined by maximum angles detected using a peak detection function.

19. A wearable device, comprising:
at least one garment configured to be worn by a user;
a plurality of sensors coupled to the at least one garment, the plurality of sensors arranged to measure a range of motion of the user; and
a wireless communication interface coupled to the at least one garment, the wireless communication interface communicatively coupled to the plurality of sensors and configured to transmit data received from the plurality of sensors.

20. The wearable device of claim 19, wherein the plurality of sensors includes a first sensor configured to be a reference sensor, and wherein the plurality of sensors includes at least one of an accelerometer, a gyroscope, and an Inertial Measurement Unit (IMU).
